(19) United States
(12) Patent Application Publication
(10) Pub. No.: US 2010/0061954 A1
(43) Pub. Date: Mar. 11, 2010

(54) HAIR TREATMENT COMPOSITIONS

(76) Inventors: Gerald Adams, Wirral (GB); Ezat Khoshdel, Wirral (GB)

Correspondence Address: UNILEVER PATENT GROUP, 800 SYLVAN AVENUE, AG West S. Wing, ENGLEWOOD CLIFFS, NJ, 07632-3100, US

(21) Appl. No.: 12/516,216

(22) PCT Filed: Nov. 2, 2007
(86) PCT No.: PCT/EP07/61810

§ 371 Date: May 26, 2009

(30) Foreign Application Priority Data
Nov. 28, 2006 (EP) EP06124936

(51) Int. Cl. A61K 8/81; A61Q 5/06

(52) U.S. Cl. 424/70.16

(57) ABSTRACT

A hair care composition comprising: i) a bimodal polymer network, comprising a first polymer with anionic character and a second polymer with cationic character, wherein the polymers form the bimodal polymer network having a molecular weight Mw below 20,000 daltons; and ii) an olefin-graft copolymer having a molecular weight Mw less than 10,000 daltons.

### FIELD OF THE INVENTION

The present invention relates to hair styling compositions.

### BACKGROUND AND PRIOR ART

There are many products on the market that provide temporary styling benefits and can readily be removed by water or shampooing. To date, the materials employed in hair care compositions to provide styling benefits have generally been natural or synthetic resins and have been applied in the form of, for example, sprays, mousses, gels and lotions. Such compositions can be used to style the hair only once and are not effective for re-styling hair once the initial style has been achieved.

Thus, there is a need for a styling composition that can be used to style and re-style the hair. Such a system requires a strong bonding action on the hair so the style is held firmly, yet the hair must not feel stiff.

The present inventors have found that certain polymer combinations can be used to style and to re-style the hair without compromising bond strength.

### DETAILED DESCRIPTION OF THE INVENTION

Accordingly the present invention relates to a hair care composition comprising:

i) a bimodal polymer network, comprising a first polymer with anionic character and a second polymer with cationic character, wherein the polymers form the bimodal polymer network having a molecular weight Mw below 20,000 daltons; and
ii) an olefin-graft copolymer having a molecular weight Mw less than 10,000 daltons.

The invention further relates to a method of styling the hair comprising the step of applying to the hair the composition described above.

### DETAILED DESCRIPTION

### Bimodal Polymer Network

Compositions of the present invention comprise a bimodal polymer. The bimodal polymer composition comprises a first polymer with anionic character and a second polymer with cationic character these polymers form an interpenetrating polymer network.

In the context of the present invention the term “Bimodal,” describes a polymer combination that include two polymers, one having anionic character and one having cationic character. An “interpenetrating polymer network,” refers to a polymer structure wherein a monomer has been polymerized in the presence of another polymer. An interpenetrating polymer network contains a polymer homogeneity that generally is not obtained from blending two polymers. The bimodal polymer network has a molecular weight Mw below 20,000 daltons, preferably from 1,000 to 20,000 daltons, more preferably from 4,000 to 12,000 daltons.

Preferably both the anionic and cationic polymers within bimodal polymer network have molecular weights Mw ranging from 1,000 to 20,000 daltons, more preferably from 4,000 to 12,000 daltons.

It is advantageous for ease of formulation and to give the required bond strengths if the molecular weights(Mw) of the cationic and anionic polymers o the bimodal polymer network are similar, ideally within 1,000 daltons of each other. It is preferred if the first polymer includes the carboxylate salt monomer unit represented by Chemical Structure I

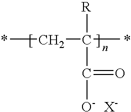

wherein R is hydrogen or a C₁ to C₆ alkyl group and X is a salt-forming cation eg. Na, K, Cs, NH4+, ethanolamine The first polymer of the bimodal polymer composition includes, may contain at least 10 weight percent of carboxylate salt monomer units. Preferably from 10 to 20 weight percent of carboxylate salt monomer units. The second polymer of the bimodal polymer network includes the cationic monomer unit represented by the chemical structure II:

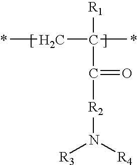

wherein R1, R3 and R4 are, independently, hydrogen or an C₁ to C₆ alkyl group and R2 is an alkyl group and R₂ is (—CH₂—)ₙ, n=1-10

Or quaternised adduct thereof as shown below:

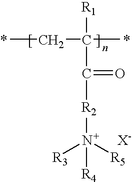

Where R₁, R₂, R₃, R₄ and R₅ are independently, hydrogen or an alkyl group and X is an anion, eg chloride, hydroxide, bromide, iodide, etc. and R₂ is (—CH₂—)ₙ, n=1-10. In one embodiment, it is preferred if the second polymer (with cationic character) includes an ammonium derivative monomer unit. The ammonium derivative monomer unit can include, but is not limited to, dialkyl amino alkyl acrylates, dialkyl amino alkyl methacrylates, quaternised adducts of dialkyl amino alkyl acrylate, quaternised adducts of dialkyl amino alkyl methacrylate, methacrylamide and esters thereof, vinyl pyrrolidone and vinyl caprolactam. In one embodiment, the ammonium derivative monomer unit is dimethylaminoethyl methacrylate or a quaternised adduct thereof.

The second polymer preferably comprises from 30 to 70 weight percent of the ammonium derivative monomer unit(s). Preferably the second polymer includes a water insoluble monomer unit. The water insoluble monomer unit can include, but is not limited to, esters of acrylate, esters of methacrylate, ethers of acrylate, ethers of methacrylate, styrenes, and alpha-methyl styrene. Most preferably the water insoluble monomer unit is butyl methacrylate. Preferably the second polymer comprises from 30 to 70, weight percent water insoluble monomer unit(s).

The second polymer can also include a water soluble monomer unit. For example, water soluble monomer units can include hydroxy functional acrylates, hydroxy functional methacrylates, and alkoxylated adducts thereof such as ethoxylated and/or propoxylated adducts thereof. In one particular embodiment, the second polymer includes a hydroxypropyl methacrylate monomer unit. Preferably the level water insoluble monomer unit(s) is from 10 to 15 weight percent o the total polymer.

The second polymer can include a cross-linking or multifunctional monomer unit. For example, the second polymer can contain a multifunctional acrylate, a multifunctional methacrylate, dially phthalate, or any other cross-linking or multifunctional monomer units known in the art. The second polymer preferably comprises up to 10 weight percent of the cross-linking or multifunctional monomer unit(s).

In some embodiments, the second polymer can include a monomer unit of anionic functionality such as, but not limited to, acrylic acid, methacrylic acid and esters thereof.

It is preferred if the second polymer includes an ammonium; derivative monomer unit, a water insoluble monomer unit, and optionally, a water soluble monomer unit (e.g. hydroxy functional acrylates, hydroxy functional methacrylates, and alkoxylated adducts thereof) and/or a cross-linking or multifunctional monomer unit.

In one embodiment, the first polymer can be present in the bimodal polymer composition in a concentration of 10 to 30 weight percent. The second polymer can be present in the bimodal polymer from 30 to 70 weight percent.

The first polymer can also include a water soluble monomer unit. For example, water soluble monomers can include hydroxy functional acrylates, hydroxy functional methacrylates, and alkoxylated adducts thereof such as ethoxylated and/or propoxylated adducts thereof. The first polymer can contain up to about 80 weight percent of the water soluble monomer unit(s).

The first polymer can include a cross-linking or multifunctional monomer unit. For example, the first polymer can contain a multifunctional acrylate, a multifunctional methacrylate, dially phthalate, or any other cross-linking or multifunctional monomer units known in the art. The first polymer can contain up to 10 weight percent of the cross-linking or multifunctional monomer unit(s).

Suitable bimodal polymer networks are described in WO 2005/087191.

Especially suitable polymers for use with the invention are those known as the Syntran PC5100 series ex Interpolymer corporation. Syntran PC5100 styrene-acrylate-amm methacrylate/bimodal polymer technology)(Mw 9200-9800) is preferred, Syntran PC5107 (polyacrylate 18&19/bimodal polymer technology)(Mw 5300-5800) is especially preferred. Compositions of the invention preferably comprise from 0.1 to 10 weight % of the total composition of the bimodal polymer network, more preferably from 1 to 6 wt %.

### Olefin Graft Polymers

The emulsion of the present invention comprises an olefin-graft copolymer, preferably the olefin-graft copolymer is semicrystalline. Olefin Graft Polymers are usually developed by grafting an amorphous functional acrylic polymer section onto a crystalline olefin backbone.

The semi-crystalline olefin copolymer can include two or more of the following monomers: ethylene, propylene, acrylic acid, methacrylic acid, melaic anhydride, crotonic acid, vinyl acetate, ethyl acrylate and similar lower C₁-C₈ esters of acrylic and methacrylic acid.

In the context of the present invention the term “semicrystalline polymer” refers to polymers that exist as viscous liquids at temperatures above the melting point of the crystals. Upon cooling, crystals nucleate and grow to fill the available volume. The reason these materials are called “semicrystalline” is that some fraction of the polymer remains un-crystallized, or, amorphous when the polymer is cooled to room temperature. The amorphous polymer becomes trapped between the growing crystals. As a result of the highly entangled nature of the polymer chains, the movement of the amorphous polymer becomes restricted.

It is advantageous if the olefin-graft copolymer is emulsified with the copolymer of acrylic acid and/or methacrylic acid and for unsaturated acidic monomer. The copolymer of the acidic monomer can include one or more of the following monomers: ethyl acrylate, butyl acrylate, 2-ethyl hexyl acrylate, butyl methacrylate, methyl methacrylate, styrene, an ethoxylated C1-C6 ester of acrylic and methacrylic acid such as hydroxpropyl methacrylate, hydroxyethyl methacrylate, hydroxylethyl acrylate, polyethylene glycol (PEG) and polypropylene glycol (PPG) modified acrylates and methacrylates with I-10 moles of ethylene oxide or propylene oxide or combination of both. The copolymer of acrylic acid can further include one or more of the following monomers: the lower amino alkyl (C1-C6) esters of methacrylic and acrylic acid, such as dimethylaminoethyl methacrylate, diethylaminoethyl methacrylate, t-butylaminoethyl methacrylate, dimethyldiallylmethacrylate as well as their quaternized adducts.

Semi-crystalline olefin polymers are commercially available from various suppliers such as Honeywell, Dupont and Sud-Chemie. Polymers of-6 this type are supplied in a pellet or flake form and must be emulsified before use.

The olefin-graft copolymer for use with the present invention has a molecule weight Mw below 10,000, more preferably below 8,000 daltons, most preferably from 3,000 to 7,000 daltons.

Examples of suitable olefin-graft polymers include syntran PC5205 a polymer of polyacrylate 15&17/olefin-graft polymer (Mw 6100 to 6300).

Compositions of the invention preferably comprise from 0.1 to 10 weight % of the total composition of the olefin-graft polymer, more preferably from 1 to 5 wt %.

The weight ratio of bimodal polymer to olefin-graft copolymer is preferably from 1:2 to 2:1

### Further Components

The hair care compositions of the present invention can be formulated into a wide variety of product types, including mousses, gels, lotions, tonics, sprays, shampoos, conditioners, rinses. Compositions of the invention comprise a cosmetically acceptable diluent or carrier. Preferably, the compositions are leave in formulations and are usually for styling hair.

### Carriers

Compositions of the present invention can comprise a carrier, or a mixture of such carriers, which are suitable for application to the hair. The carriers are present at from about 0.5% to about 99.5%, preferably from about 5.0% to about 99.5%, more preferably from about 10.0% to about 98.0%, by weight of the composition. As used herein, the phrase “suitable for application to hair” means that the carrier does not damage or negatively affect the aesthetics of hair or cause irritation to the underlying skin.

Carriers suitable for use with hair care compositions of the present invention include, for example, those used in the formulation of hair sprays, mousses, tonics, gels, shampoos, conditioners, and rinses. The choice of appropriate carrier will also depend on the particular polymer to be used, and whether the product formulated is meant to be left on the surface to which it is applied (e.g., hair spray, mousse, tonic, or gel) or rinsed off after use (e.g., shampoo, conditioner, rinse). The carriers used herein can include a wide range of components conventionally used in hair care compositions. The carriers can contain a solvent to dissolve or disperse the particular polymer being used, with water, the C₁-C₆ alcohols, lower alkyl acetate and mixtures thereof being preferred. The carriers can also contain a wide variety of additional materials such as acetone, hydrocarbons (such as isobutane, hexane, decene), halogenated hydrocarbons (such as Freons) and volatile silicones such as cyclomethicone.

When the hair care composition is a hair spray, tonic, gel, or mousse the preferred solvents include water, ethanol, volatile silicone derivatives, and mixtures thereof. The solvents used in such mixtures may be miscible or immiscible with each other. Mousses and aerosol hair sprays can also utilise any of the conventional propellants to deliver the material as a foam (in the case of a mousse) or as a fine, uniform spray (in the case of an aerosol hair spray). Examples of suitable propellants include materials such as trichlorofluoromethane, dichlorodifluoromethane, difluoroethane, dimethylether, propane, n-butane or isobutane. A tonic or hair spray product having a low viscosity may also utilise an emulsifying agent. Examples of suitable emulsifying agents include nonionic, cationic, anionic surfactants, or mixtures thereof. If such an emulsifying agent is used, it is preferably present at a level of from about 0.01% to about 7.5% by weight based on total weight of the composition. The level of propellant can be adjusted as desired but is generally from about 3% to about 30% by weight based on total weight for mousse compositions and from about 15% to about 50% by weight based on total weight for aerosol hair spray compositions.

Suitable spray containers are well known in the art and include conventional, non-aerosol pump sprays i.e., “atomisers”, aerosol containers or cans having a propellant, as described above, and also pump aerosol containers utilising compressed air as the propellant.

Where the hair care compositions are conditioners and rinses, the carrier can include a wide variety of conditioning materials. Where the hair care compositions are shampoos, the carrier can include, for example, surfactants, suspending agents, and thickeners. Hair styling creams or gels also typically contain a structurant or thickener, typically in an amount of from 0.01% to 10% by weight.

The carrier can be in a wide variety of forms. For example, emulsion carriers, including oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurised aerosol containers using conventional propellants. These carriers can also be delivered in the form of a mousse. Other suitable topical carriers include anhydrous liquid solvents such as oils, alcohols, and silicones (e.g., mineral oil, ethanol, isopropanol, dimethicone, cyclomethicone, and the like); aqueous-based single phase liquid solvents (e.g., hydro-alcoholic solvent systems); and thickened versions of these anhydrous and aqueous-based single phase solvents (e.g., where the viscosity of the solvent has been increased to form a solid or semi-solid by the addition of appropriate gums, resins, waxes, polymers, salts, and the like).

### Additional Components

A wide variety of additional components can be employed in compositions according to the present invention. Examples include the following:
further hair styling polymers for hair styling compositions such as hair sprays, gels, and mousses. Hair styling polymers are well known articles of commerce and many such polymers are available commercially which contain moieties, which render the polymers cationic, anionic, amphoteric or nonionic in nature. The polymers may be synthetic or naturally derived.
sunscreening agents
anti-dandruff actives
hair conditioning agents such as hydrocarbons, silicone fluids, and cationic materials.
surfactants for hair shampoo and conditioner compositions. Surfactants useful in compositions of the present invention include anionic, nonionic, cationic, zwitterionic and amphoteric surfactants.
vitamins and derivatives thereof (e.g., ascorbic acid, vitamin E, tocopheryl acetate, retinoic acid, retinol, retinoids, and the like).
cationic polymers (e.g., cationic guar gum derivatives such as guar hydroxypropyltrimonium chloride and hydroxypropyl guar hydroxypropyltrimonium chloride, available as the Jaguar® series from Rhone-Poulenc).
preservatives, antioxidants, chelators and sequestrants; and aesthetic components such as fragrances, colourings, hair nutrients and essential oils.

The following non-limiting Examples further illustrate the preferred embodiments of the invention. All percentages referred to are by weight based on total weight unless otherwise indicated.

### EXAMPLES

### Materials

| | | % | |
|---|---|---|---|
| Name | Composition/Technology | Solids | Mw(daltons) |
| Syntran | Polyacrylate 15&17/ | 30 | 6100-6200 |
| PC5205 | olefin graft polymer | | |
| | technology | | |
| Syntran | Polyacrylate | 30 | 5300-5800 |
| PC5107 | 18&19/Bimodal polymer | | |
| | technology | | |
| Syntran | Polyacrylate 15 (PE | 38 | 11,000-12,000 |
| PC5208 | backbone)/olefin graft | | |
| | polymer technology | | |
| Syntran | Styrene-acrylates-amm | 25 | 9200-9800 |
| PC5100 | methacrylate | | |
| | copolymer/Bimodal polymer | | |
| | technology | | |

### Bond Strength

The bond strength of 2% aqueous solid solutions were measured using the Diastron MTT 600 by forming a polymer junction on perpendicular hair fibres and allowing to dry. Instrument parameters were: (% extension=100, gauge force=0.5, threshold=20, rate=70 mm/min, max force=200 g)

Bimodal Networks or Olefin-Grafted Polymers with No Blending

Measurements at 50% relative humidity

| Name | Bond strength (g) | % extension |
|---|---|---|
| Syntran | 8.55 (+/−2.2) | 1.85 (+/−0.5) |
| PC5205 | | |
| Syntran | 22.97 (+/−2.7) | 4.43 (+/−1.4) |
| PC5107 | | |
| Syntran | 13.23 (+/−5.1) | 2.55 (+/−0.7) |
| PC5208 | | |
| Syntran | 5.75 (+/−1.9) | 1.45 (+/−0.8) |
| PC5100 | | |

### Bimodal Network Polymers/Olefin-Graft Copolymers 50:50 Blends

Measurements at 50% relative humidity

| Name | Bond strength (g) | % extension |
|---|---|---|
| Syntran | 31.5 (+/−2.6) | 4.00 (+/−0.9) |
| PC5107:Syntran | | |
| PC5205 | | |
| Syntran | 14.9 (+/−2.4) | 2.47 (+/−0.9) |
| PC5100:Syntran | | |
| PC5205 | | |
| Syntran | 6.73 (+/−3) | 3.43 (+/−1.2) |
| PC5107:Syntran | | |
| PC5208 | | |
| Syntran | 7.97 (+/−2.3) | 2.20 (+/−0.4) |
| PC5100:Syntran | | |
| PC5208 | | |

Thus those blends comprising an olefin-graft having a molecular weight less than 10,000 a molecular weight have the greatest bond strength without a detrimental effect on elasticity.

The materials used in the examples include the following:

| Material | Supplier | Function |
|---|---|---|
| Silicone emulsion | Dow Corning | conditioning |
| X2 1787 ™ | | |
| VOLPO CS 50 ™ | Croda Chemicals | surfactant |
| Sepicide LD ™ | Seppic | preservative |
| Cremophor RH410 ™ | BASF | stabiliser |

Ethanol is SD Alcohol 40-B (92% active)

### Example 1

### A Styling Mousse

| Material | % in product (w/w) |
|---|---|
| Silicone Emulsion X2 1787 | 1.2 |
| Bimodal + Olefin Graft | 1.5 |
| Syntran PC100:Syntran PC 5205 | |
| 50:50 blends | |
| VOLPO CS 50 | 0.3 |
| Sepicide LD | 0.4 |
| Cremophor RH410 | 0.2 |
| Ethanol | 7.5 |
| CAP 40 | 8.0 |
| Perfume | 0.2 |
| Water | to 100% |

### Example 2

### A Pump Spray

| Material | % in product (w/w) |
|---|---|
| Bimodal + Olefin Graft | 3.0 |
| Syntran PC100:Syntran PC 5205 | |
| 50:50 blends | |
| Silicone DC200 | 0.09 |
| Silwet L7602 | 0.09 |
| CAP 40 | 35.0 |
| Ethanol | 60.0 |
| Perfume | 0.10 |
| Water | to 100% |

### Example 3

### A Pump Spray

| Material | % w/w |
|---|---|
| Ethanol | 60.0 |
| Bimodal + Olefin Graft | 3.5 |
| Syntran PC100:Syntran PC 5205 | |
| 50:50 blends | |
| Silwet L-720 | 0.3 |
| Silicone DC24S | 0.15 |
| Fragrance | 0.3 |
| Water | to 100% |

### Example 4

### A Styling Gel

| Material | % w/w |
|---|---|
| Bimodal + Olefin Graft | 3.8 |
| Syntran PC100:Syntran PC 5205 | |
| 50:50 blends | |
| Carbopol 980 | 0.4 |
| Water | to 100% |
| Sepicide LD | 0.4 |
| Sodium hydroxide (8% 2M) | 0.1 |
| Ethanol | 10.0 |
| Cremaphor RH410 | 0.4 |
| Jaguar HP-105 | 0.2 |
| Perfume | 0.15 |

### Example 5

### A 55% voc Propelled Aerosol Composition

| Material | % w/w |
|---|---|
| Bimodal + Olefin Graft | 3.75 |
| Syntran PC100:Syntran PC 5205 | |
| 50:50 blends | |
| Silicone Fluid 245 | 0.20 |
| Fragrance | 0.32 |
| Ethanol | 19.53 |
| Dimethyl ether | 35.00 |
| Sodium benzoate | 0.26 |
| Cyclohexylamine | 0.21 |
| Water | to 100% |

### Example 6

### A 55% voc Pump Hairspray Composition is Formulated as Follows

| Material | % w/w |
|---|---|
| Bimodal + Olefin Graft | 3.75 |
| Syntran PC100:Syntran PC 5205 | |
| 50:50 blends | |
| Cyclopentasiloxane (99% active) | 0.15 |
| Benzophenone 4 | 0.0001 |
| Fragrance | 0.25 |
| Ethanol | 58.00 |
| Water | to 100% |

1. A hair care composition comprising:
i) a bimodal polymer network, comprising a first polymer with anionic character and a second polymer with cationic character, wherein the polymers form the bimodal polymer network having a molecular weight Mw below 20,000 daltons; and
ii) an olefin-graft copolymer having a molecular weight Mw less than 10,000 daltons.

2. A hair care composition according to claim 1 wherein the first polymer within the bimodal polymer network has a molecular weight Mw ranging from 4,000 to 12,000 daltons.

3. A hair care composition according to claim 1 wherein the second polymer of the bimodal polymer network has a molecular weight Mw ranging from 4,000 to 12,000 daltons.

4. A hair care composition according to claim 1 wherein the olefin-graft copolymer has a molecule weight Mw below 8,000 daltons.

5. A hair care composition according to claim 1 wherein the first polymer of the bimodal polymer network includes the following carboxylate salt monomer unit: 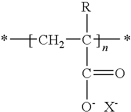
wherein R is hydrogen or an alkyl group and X is a salt-forming cation.

6. A hair care composition according to claim 1 in which the first polymer of the bimodal polymer network contains at least about 10 weight percent of carboxylate salt monomer units.

7. A hair care composition according to claim 1 in which the second polymer of the bimodal polymer network comprises an ammonium derivative monomer unit.

8. A hair care composition according to claim 1 in which the ammonium derivative monomer of the second polymer of the bimodal polymer network is dimethylaminoethyl methacrylate or a quaternized adduct thereof.

9. A hair care composition according to claim 1 in which the second polymer of the bimodal polymer network comprises a water insoluble monomer unit.

10. A hair care composition according to claim 1 in which the second polymer of the bimodal polymer network comprises up to about 10 weight percent of a cross-linking or multifunctional monomer unit.

11. A hair care composition according to claim 1 wherein the olefin-graft copolymer comprises two or more of the following monomers: ethylene, propylene, acrylic acid, methacrylic acid, melaic anhydride, crotonic acid, vinyl acetate, ethyl acrylate and C1-C8 esters of acrylic and methacrylic acid.

12. A hair care composition according to claim 1 in which the weight ratio of bimodal polymer to olefin-graft polymer is from 1:2 to 2:1.

13. A method of styling the hair comprising the step of applying to the hair the composition as described in claim 1.
